(19) United States
(12) Patent Application Publication
(10) Pub. No.: US 2024/0271066 A1
(43) Pub. Date: Aug. 15, 2024

(54) MULTI-LAYERED MODULAR ORGAN-ON-A-CHIP PLATFORM

(71) Applicant: RAMOT AT TEL AVIV UNIVERSITY LTD., Tel Aviv (IL)

(72) Inventors: Ben Meir MAOZ, Tel Aviv (IL); Anna HERLAND, Tel Aviv (IL); Shay DIVALD, Tel Aviv (IL); Yuval OLIEL, Tel Aviv (IL)

(21) Appl. No.: 18/570,211

(22) PCT Filed: Jun. 29, 2022
(86) PCT No.: PCT/IL2022/050698

§ 371 Date: Dec. 14, 2023

Related U.S. Application Data
(60) Provisional application No. 63/217,356, filed on Jul. 1, 2021.

(51) Int. Cl. C12M 3/00; C12M 1/00; C12M 1/12; C12M 1/34; C12M 3/06

(52) U.S. Cl. CPC C12M 21/08; C12M 23/16; C12M 23/34; C12M 23/42; C12M 25/04; C12M 29/06; C12M 41/46

(57) ABSTRACT

Provided herein is a modular organ-on-a-chip platform, configured for cultivating cells, tissues, organoids and organelles and capable of performing high throughput analyses, including pharmacokinetic evaluations.

### FIELD OF THE INVENTION

Provided herein is a modular organ-on-a-chip platform, configured for cultivating cells, organelles and organoids, and capable of performing high throughput analyses, including pharmacokinetic evaluations.

### BACKGROUND

Organs-on-chips platform enables to study diseases and obtain pharmacokinetic evaluations for potential drugs, in vitro, in a system that resembles cellular and organ interaction of a living animal or human subject.

Various organ-on-a-chip systems are known in the art, for example, those disclosed in U.S. Pat. Nos. 10,119,622; 10,078,075; 9,725,687; US 2017/0370908; WO 2019/222872; WO 2018/094003; EP 3525584; EP 2730644; CN 109070082; CN 106811413; DK 2712918; Novak et al. (Nature Biomedical Engineering, 4, 407-420, 2020) and Herland et al. (Nature Biomedical Engineering, 4, 421-436, 2020).

An organ-on-a-chip typically includes two types of cells, cultured on both sides of a membrane, namely, vascular endothelial cells and organ-specific cells, wherein each chip is individually perfused with blood equivalent media and tissue culture medium, for culturing the vascular endothelial cells and organ-specific cells, respectively. Organ-on-a-chip systems include several chips, and respective conduits and pumps, which render such systems complex, difficult to construct, and often not clinically relevant.

There is an unmet need for a modular organ-on-a-chip, cost effective, which is easy to assemble and disassemble, which allows the study of various cellular interactions as well as providing reliable pharmacokinetic data.

### SUMMARY

According to some embodiments, there are provided herein advantageous modular organ-on-a-chip devices and systems, which are cost effective, easy to assemble, and which allow versatile settings to facilitate the study of cellular behavior and inter-cellular interactions, under various conditions. According to some embodiments, the systems and devices disclosed herein may be in the form of a cartridge configured to include one or more (a plurality) of organ-on-a-chip units, also termed herein organ chips, which are fluidly connected under a single pool of blood (or blood equivalent medium). Optionally, the cartridge may comprise three main components: one or more organ chips configured for culturing cells, organelles and/or organoids on a membrane included therein, a receptacle configured to contain blood (or blood equivalent medium), and a cover layer which may be configured to hold the one or more organ chips such that their bottom part which may include a membrane with cells (including such cells as, but not limited to: vascular endothelial cells, cancerous cells, epithelial cells, alveolar epithelial cells, immune cells, enterocytes, and goblet cells, cardiac cells, muscle cells, adiposes cells, skin cells, hepatic cells, spheroids, stem cell-derived organoids, and the like, or any combinations thereof) facing the receptacle, may be immersed in the receptacle.

Advantageously, all the organ chips within a cartridge may receive a direct supply of blood, or blood equivalent medium, by being immersed in a receptacle containing these fluids. Optionally, this configuration may be devoid of excess conduits, pipes and the like, and thus may reduce potential blockages, contamination and other well-known complications associated with pipe systems.

Moreover, advantageously, the design of the cartridge may enable easily adding thereto, or removing therefrom, organ chips, without complicated connection/disconnection to pipes and pumps, due to the use of a mutual source of blood/blood equivalent in a single container. Optionally, the container may also contain reservoirs in fluid connection with the fluids in the container, which enables to easily derive blood/blood equivalent samples and may not require special pipe openings or pipe stubbing.

Furthermore, each organ chip may include a window that enables a clear and direct view of the cells cultured therein. Optionally, the cells culture on the organ chips may be monitored at all times, where the images obtained through the window may be undistorted and comprehensive.

In some embodiments, there is provided a cartridge for culturing tissues (organs)-on-a-chip, comprising:
a. a receptacle configured to contain fluids, the receptacle comprises at least one receptacle inlet configured to deliver a first fluid into the receptacle, at least one receptacle outlet configured to withdraw fluids from the receptable;
b. at least one organ chip having a bottom face configured to be in fluid communication with fluids within the receptacle, the organ chip comprising
   i. a membrane located at the bottom face of the at least one organ chip, the membrane having an upper side and a lower side each configured for cell, tissue or organoid culture, wherein the membrane is permeable to fluids and wherein the upper side of the membrane is facing the top face of the organ chip;
   ii. an inlet conduit having an external inlet protruding through the top face of the organ chip, wherein the inlet conduit is configured to deliver a second fluid into the organ chip;
   iii. an outlet conduit having an external outlet protruding through the top face of the organ chip, wherein the outlet conduit is configured to withdraw fluids from the organ chip; and
c. a cover layer configured to cover the receptacle, the cover layer comprising at least one slot configured to hold the at least one organ chip such that the bottom face of the organ chip and the lower side of the membrane are immersed in the receptacle and the external outlet and inlet protrude outside the cartridge, throughout the cover layer.

In some embodiments, the first fluid may be blood or blood equivalent medium, and the second fluid may be a tissue culture medium.

In some embodiments, the first fluid may be blood, blood equivalent medium or tissue culture medium. In some embodiments, the second fluid may be a tissue culture medium, body fluid other than blood, or an artificial equivalent thereof.

Optionally, the blood, blood equivalent and/or tissue culture medium may comprise additives, such as hormones, drugs, vitamins, amino acids, buffers, etc., or any combination thereof.

In some embodiments, the receptacle may further comprise at least one reservoir in fluid communication with the fluids within the receptacle. Optionally, the cover layer may further comprise at least one reservoir opening configured to be positioned above the at least one reservoir. Optionally, thereby enabling withdrawal of fluids from the receptacle through the at least one reservoir opening.

In some embodiments, the receptacle may further comprise at least one fluid impermeable partitioning unit creating a barrier that may prevent flow of the fluids out of the receptacle. Optionally, the partitioning units may comprise flexible members and/or rigid and/or semi-rigid dams. Optionally, the partitioning units may be held in place by partitioning holders.

In some embodiments, the cartridge may comprise a plurality of organ chips. Optionally, the cover layer may include a plurality of slots, each configured to hold one organ chip from the plurality of organ chips.

In some embodiments, the cartridge may comprise a plurality of reservoirs. Optionally, the cover layer may comprise a plurality of reservoir openings. Optionally, each reservoir opening may be configured to be positioned above one reservoir from the plurality of reservoirs.

In some embodiments, the top face of the organ chip may comprise an opening configured to enable view of the cells and/or organelles cultured on the top side of the membrane.

In some embodiments, the receptacle may comprise a plurality of receptacle inlets and/or a plurality of receptacle outlets.

In some embodiments, there is provided an organ-on-a-chip system, comprising at least one cartridge, a container containing the first fluid fluidly connected to the receptacle through the at least one receptacle inlet; a container containing the second fluid fluidly connected to the organ chip through the external inlet; a first withdrawal pump configured to withdraw fluids from the receptacle through the at least one receptacle outlets; and optionally, a second withdrawal pump configured to withdraw fluids from the organ chip through the external outlet.

In some embodiments, said fluidly connected may comprise fluidly connected through a pump.

In some embodiments, the at least one cartridge may comprise a plurality of organ chips. Optionally, the organ chips may be the same and/or different. Optionally, the organ chips may be fluidly connected in series and/or in parallel. In some embodiments, the fluid connection between the organ chips may be predetermined. In some embodiments, the fluid connection between the organ chips may be adjusted prior to and/or during an experiment.

In some embodiments, the at least one cartridge may comprise a single organ chip.

In some embodiments there is provided a method for assembling a cartridge for culturing tissues (organs)-on-a-chip, the method comprising the steps of:
a. providing a receptacle configured to contain fluids, the receptacle comprises at least one receptacle inlet configured to deliver a first fluid into the receptacle, at least one receptacle outlet configured to withdraw fluids from the receptable;
b. providing a first container containing a first fluid and a second container containing a second fluid;
c. fluidly connecting the first container to the at least one receptacle inlet, and fluidly connecting the at least one receptacle inlet to a first drainage container;
d. providing at least one organ chip having a bottom face configured to be in fluid communication with fluids within the receptacle, the organ chip comprising
   i. a membrane located at the bottom face of the at least one organ chip, the membrane having an upper side and a lower side each configured for cell or organelle culture, wherein the membrane is permeable to fluids and wherein the upper side of the membrane is facing the top face of the organ chip;
   ii. an inlet conduit having an external inlet protruding through the top face of the organ chip, wherein the inlet conduit is configured to deliver a second fluid into the organ chip;
   iii. an outlet conduit having an external outlet protruding through the top face of the organ chip, wherein the outlet conduit is configured to withdraw fluids from the organ chip,
e. covering the receptacle with a cover layer comprising at least one slot configured to hold the at least one organ chip;
f. positioning the at least one organ chip in the at least one slot such that the bottom part of the membrane is immersed in the fluids within the receptacle and the external outlet and inlet protrude outside the cartridge, through the cover layer; and
g. fluidly connecting the second container to the external inlet, and fluidly connecting the external outlet to a second drainage container,
wherein said fluidly connecting comprises connecting through conduits and pumps to initiate flow-in through the at least one receptacle inlet and the external inlet and flow-out through the at least one receptacle outlet and the external outlet.

In some embodiments, the receptacle may further comprise at least one fluid impermeable partitioning unit creating a barrier that prevents flow of the fluids out of the receptacle. In some embodiments, the partitioning units may comprise rigid and/or flexible partitions. Optionally, the rigid partitions may be held in place by one or more partitioning holders. Optionally, the partitions may provide a channel through which a first fluid may pass. Optionally, the partitions may be removably positioned to provide a variety of configurations. Optionally, the partitions may be fixed in position, e.g., by molding, gluing, baking, etc. Optionally, the partitions may be configured in parallel and/or in series, e.g., to mimic the natural order of organs and/or organelles in the body.

In an embodiment, a lid or cover sheet may be fixed to the cover layer and/or base layer over each organ chip and/or chip opening to seal the system. Optionally, the cover sheet may be fixed in place, for example by one or more screws. Optionally, the screws may pass through corresponding holes in the cover sheet and/or cover sheet and/or base layer. Optionally, the screws may be tightened to prevent leakage of the fluids.

In some embodiments, the receptacle may further comprise at least one reservoir in fluid communication with the fluids within the receptacle. Optionally, the cover layer may further comprise at least one reservoir opening positioned above the at least one reservoir. Optionally, thereby enabling withdrawal of fluids from the receptacle through the at least one reservoir opening.

In some embodiments, the method may further comprise the step of adding to the first container a pharmaceutical composition, comprising at least one pharmaceutically active ingredient.

In some embodiments, the method may further comprise the step of withdrawing a sample of fluids from the at least one reservoir, optionally periodically, and/or monitoring the amount or activity of at least one component in the sample.

In some embodiments, the at least one component may be the at least one pharmaceutically active ingredient, wherein periodical monitoring the amount thereof may be further utilized for pharmacokinetic evaluation.

According to some embodiments, there is provided an organ chip having a bottom face configured to be in fluid communication with fluids within a receptacle, the organ chip comprising: i) a membrane located at the bottom face of the organ chip, the membrane having an upper side and a lower side each configured for cell, tissue or organoid culture, wherein the membrane is permeable to fluids and wherein the upper side of the membrane faces the top face of the organ chip; (ii) an inlet conduit having an external inlet protruding through the top face of the organ chip, wherein the inlet conduit is configured to deliver a second fluid into the organ chip; and (iii) an outlet conduit having an external outlet protruding through the top face of the organ chip, wherein the outlet conduit is configured to withdraw fluids from the organ chip.

According to some embodiments, the second fluid may be a tissue culture medium, body fluid other than blood, or an artificial equivalent thereof. According to some embodiments, the organ chip may be for use with a cartridge for culturing organ on a chip.

According to some embodiments, the organ chip may be for use with an organ-on-a-chip system as disclosed herein.

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1 represents a system for culturing organs-on-a-chip, in accordance with some embodiments.

FIG. 2 represents a system for culturing organs-on-a-chip, in accordance with some embodiments.

FIGS. 3A-3C represent the receptacle of the cartridge, in accordance with some embodiments.

FIG. 4 represents the cover layer, in accordance with some embodiments.

FIG. 5 represents the cover layer, in accordance with some embodiments.

FIG. 6 represents the base layer, in accordance with some embodiments.

FIG. 7 represents the base layer with flexible members, in accordance with some embodiments.

FIGS. 8A-8C represent the organ chip of the cartridge, in accordance with some embodiments.

FIGS. 9A-9B represent the organ chip of the cartridge, in accordance with some embodiments.

FIG. 10 represents a flow diagram demonstrating an organs-on-a-chip system, in accordance with some embodiments.

FIGS. 11A-11B represents uses of an organs-on-a-chip system, in accordance with some embodiments.

FIG. 12 represents an image of cell culture grown on the upper side of membrane of an organ chip within a perfused cartridge.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

In some embodiments, there is provided a cartridge for culturing organs-on-a-chip, comprising:
a. a receptacle configured to contain fluids, the receptacle comprises at least one receptacle inlet configured to deliver a first fluid into the receptacle, at least one receptacle outlet configured to withdraw fluids from the receptable;
b. at least one organ chip having a bottom face configured to be in fluid communication with fluids within the receptacle and a top face, the organ chip comprising
   i. a membrane located at the bottom face of the at least one organ chip, the membrane may have an upper side and a lower side each configured for cell or organelle culture, wherein the membrane may be permeable to fluids and wherein the upper side of the membrane is facing the top face of the organ chip;
   ii. an inlet conduit having an external inlet protruding through the top face of the organ chip, wherein the inlet conduit may be configured to deliver a second fluid into the organ chip;
   iii. an outlet conduit having an external outlet protruding through the top face of the organ chip, wherein the outlet conduit may be configured to withdraw fluids from the organ chip; and
c. a cover layer configured to cover the receptacle, the cover layer may comprise at least one slot configured to hold the at least one organ chip such that the bottom face of the organ chip and the lower side of the membrane may be immersed in the receptacle and the external outlet and inlet may protrude outside the cartridge, throughout the cover layer.

In some embodiments, the organ chip may have a bottom face configured to be in fluid communication with fluids within a receptacle, the organ chip comprising:
i. a membrane located at the bottom face of the organ chip, the membrane having an upper side and a lower side each configured for cell, tissue or organoid culture, wherein the membrane may be permeable to fluids and wherein the upper side of the membrane faces the top face of the organ chip;
ii. an inlet conduit having an external inlet protruding through the top face of the organ chip, wherein the inlet conduit may be configured to deliver a second fluid into the organ chip;
iii. an outlet conduit having an external outlet protruding through the top face of the organ chip, wherein the outlet conduit may be configured to withdraw fluids from the organ chip.

In some embodiments, the membrane may be at least partially flexible, and/or porous and/or gas impermeable. In some embodiments, the membrane may be a synthetic membrane. In some embodiments, the membrane may be a synthetic gas permeable polymeric membrane. In some embodiments, the polymeric membrane may include cellulose acetate (CA), polyacrylonitrile (PAN), polyimide, polycarbonate (PC), polyethylene (PE), polypropylene (PP), polytetrafluoroethylene (PTFE) or polyvinylidene fluoride (PVDF). In some embodiments, the membrane may be a native membrane derived from native tissue, such as, a membrane derived from cellular barriers (e.g., endothelia cell barrier, epithelial membranes, mucous membranes, serous membranes, connective tissue membranes, synovial membranes, meninges, etc., or any combination thereof. In some embodiments, the membrane may be a synthetic membrane coated with physiological matter, such as, laminin, extracellular matrix and type IV collagen, etc.

In some embodiments, the cover layer and/or cover sheet may be configured to reversibly cover the receptacle. In some embodiments, the cover layer may be configured to cover the receptacle while allowing gas (e.g., air, CO₂, etc.) exchange between the receptacle and the environment.

In some embodiments, each slot in the cover may be configured to hold in place an organ chip. Optionally, by holding an organ chip in place, the chip may be stabilized and/or prevented from sliding and/or floating and/or moving back and forth when immersed in the receptacle's fluids. Optionally, the cover may not prevent and/or hamper removal of the chip from the cartridge. Optionally, at any given moment a chip held by the cover layer, within the cartridge may be removed therefrom, e.g., by lifting up the chip perpendicularly to the plane of the cover layer.

In an embodiment, a cover sheet may be fixed to the cover layer over each organ chip and/or chip opening to seal the system. Optionally, the cover sheet may press and/or hold the organ chip down so that the membrane may be immersed in a fluid in the receptacle. Optionally, the cover sheet may be fixed in place, for example by one or more screws. Optionally, the screws may pass through corresponding holes in the cover sheet and/or cover layer and/or base layer. Optionally, the screws may be tightened to prevent leakage of the fluid.

In some embodiments, the first fluid delivered to the receptacle may be fresh first fluid. In some embodiments, the fresh first fluid may be fresh blood. In some embodiments, the fresh first fluid may be fresh blood equivalent medium. In some embodiments, the first fluid may be tissue culture medium or an equivalent thereof. In some embodiments, the first fluid may be body fluid other than blood, including a synthetic equivalent thereof. Optionally, the blood, blood equivalent and/or tissue culture medium may comprise additives, such as hormones, drugs, vitamins, sera, cytokines, antibiotics, growth factors, amino acids, pH adjusters, buffers, and the like, or any combination thereof.

The term ‘fresh’ as used herein refers to un-used fluids, and specifically, to sterile fluids delivered from a sterile container and/or other source which may be intended to be used for tissue culture for the first time.

In some embodiments, the second fluid may be a tissue culture medium. In some embodiments, the second fluid may be tissue culture medium or an equivalent thereof. In some embodiments, the second fluid may be a body fluid other than blood, including a synthetic equivalent thereof.

In some embodiments, the second fluid may have further supplements, including, but not limited to, drugs, substances, small molecules, amino acids, buffers, vitamins, sera, cytokines, antibiotics, growth factors, hormones, and the like, or any combination thereof. Optionally, the choice of tissue culture medium and corresponding supplements may depend on the cells or organelles cultured on the upper side of the membrane.

The term ‘fluids’ as used herein refers to the fluids within the receptacle, which contain fresh first fluid, fresh second fluid and circulating (used) second fluid that diffused through the membrane from the upper side of the membrane to the receptacle. Optionally, the fluids may also contain cellular debris and particles (molecules, intracellular matrix components) discharged from the cell cultured on both sides of the membrane.

In some embodiments, the term “culturing organ on a Chip” encompass culturing cells, tissues, organoids and/or organelles, on an organ chip as disclosed herein.

In some embodiments, the receptacle may further comprise at least one fluid impermeable partitioning unit creating a barrier that prevents flow of the fluids out of the receptacle. In some embodiments, the partitioning units may comprise rigid and/or flexible partitions. Optionally, the rigid partitions may be held in place by one or more partitioning holders. Optionally, the partitions may provide a channel through which the first fluid may pass. Optionally, the partitions may be removably positioned to provide a variety of configurations. Optionally, the partitions may be fixed in position, e.g., by molding, gluing, baking, etc. Optionally, the partitions may be molded as part of the receptacle. Optionally, the partitions may be removably positioned to provide a variety of configurations. Optionally, the partitions may be configured in parallel and/or in series, e.g., to mimic the natural order of organs and/or organelles in the body. For example, partitioning units may be positioned to provide a channel for the fluid below the membrane of an organ chip, such that the fluids flowing in the receptacle flow only between the barriers.

In some embodiments, the plurality of partitioning holders may be any type of holders that can hold a partition tightly, in order to create a barrier blocking the flow of fluids therethrough. Optionally, the holders may be, for example, in the form of clips, magnets, or slits in a panel and/or in the receptacle wall.

In some embodiments, the fluid impermeable partitioning units may be configured to act as local dams, which block the flow of fluids in the receptacle, thereby regulating the flow within the receptacle. For example, a partitioning unit may be positioned between an organ chip and a wall of the receptacle, such that the fluids flowing in the receptacle will make a detour around the barrier.

In an embodiment, the receptacle may be molded onto a base layer. Optionally, the receptacle may form channels, for example by attaching (e.g., by gluing, baking, melding, binding, etc.) the partitions onto the base layer, thereby forming a receptacle. Optionally, the partitions may be sandwiched between a base layer and a cover layer. Optionally, this sandwiching may be tight to prevent leakage of the fluid passing through the receptacle. For example, screws passing through corresponding holes in the cover layer and/or the base layer may be used to adjust the tightness.

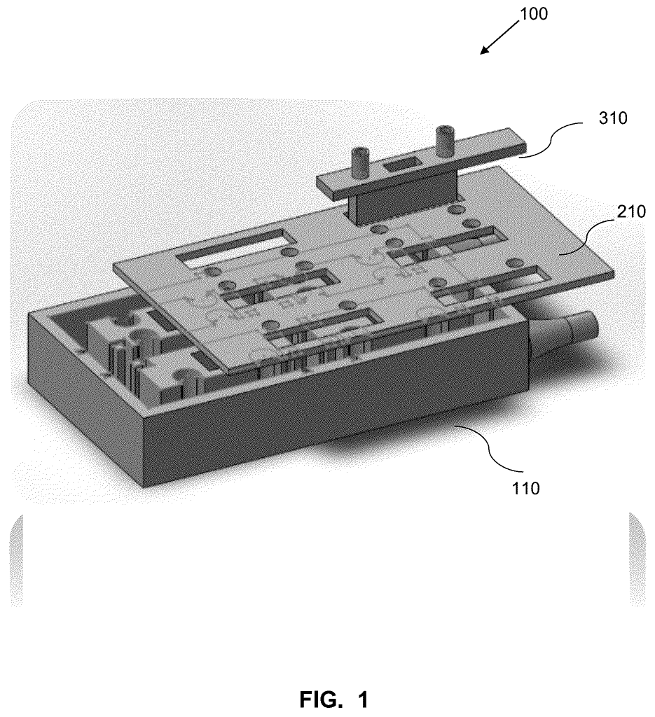

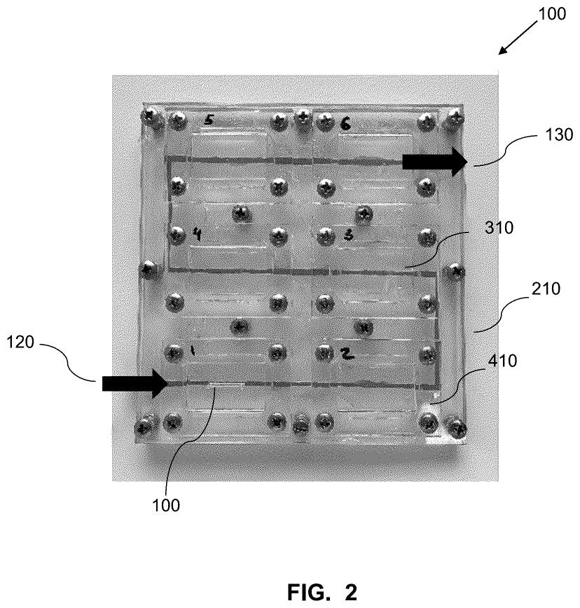

Reference is now made to FIGS. 1-5. FIG. 1 constitutes a perspective view of cartridge (device) 100 for culturing organs-on-a-chip, according to some embodiments. Optionally, cartridge 100 may include three main components: a receptacle 110, a cover layer 210 and an organ chip 310, optionally, orderly arranged one on top of the other. FIG. 2 constitutes a top view of a cartridge (device) 100 for culturing organs-on-a-chip, according to some embodiments. Optionally, cartridge 100 in FIG. 2, may include three main components: a receptacle 440, a cover layer 210 and an organ chip 310, optionally, orderly arranged one on top of the other.

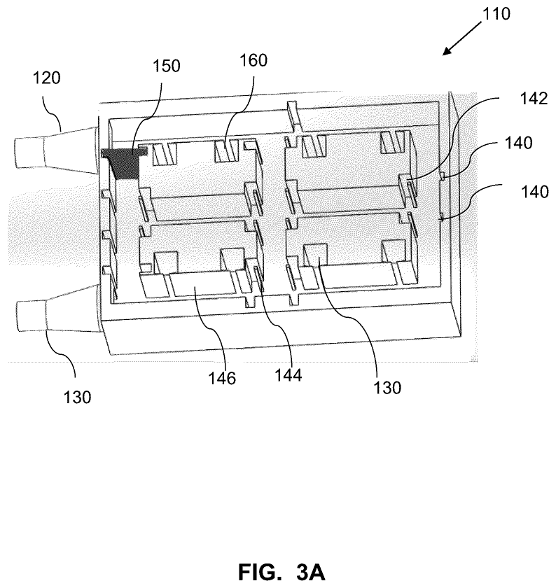

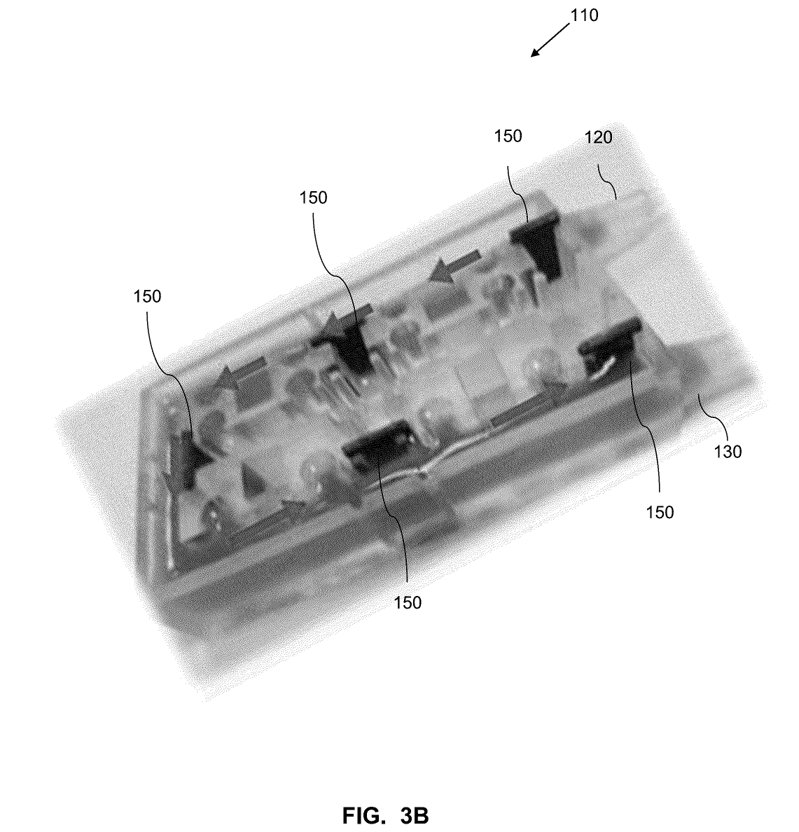

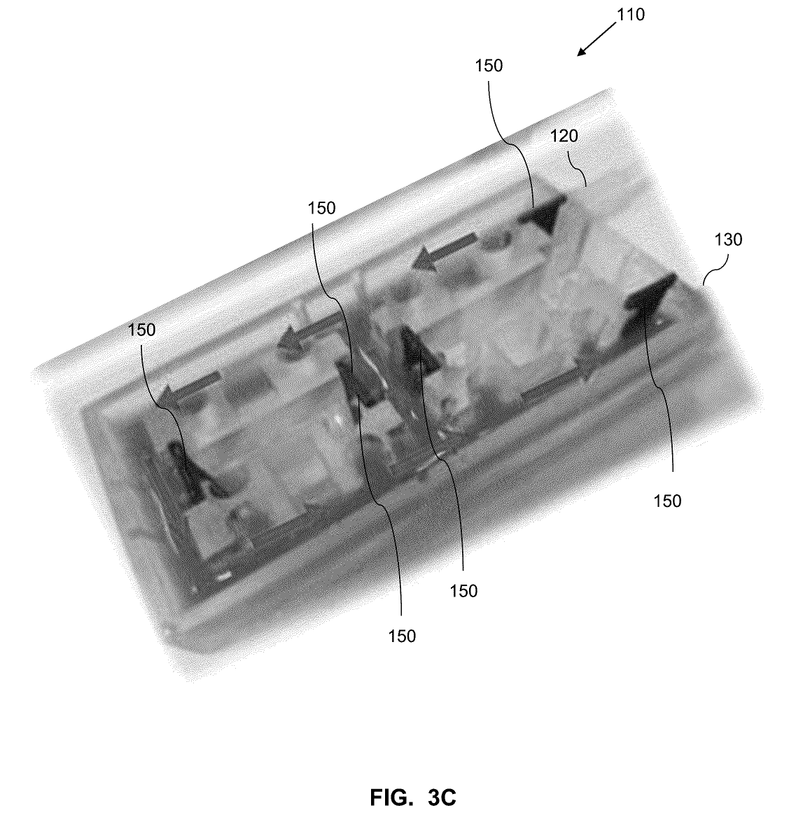

FIGS. 3A-3C constitute perspective views of receptacle 110, according to some embodiments. In some embodiments, receptacle 110 may be in the form of a basin or a container having a basis surrounded by (four) walls, where receptacle 110 may be configured to hold therewithin fluids. Receptacle 110 may include an inlet 120 and an outlet 130 configured to deliver and withdraw fluids, respectively, from receptacle 110. Receptacle 110 may further include a plurality of partitioning holders 140 configured to hold partitioning units 150 which may be fluid impermeable.

In some embodiments, receptacle 110 may further include at least one partitioning unit 150, configured to form a barrier which may be capable of preventing flow of the fluids in the receptacle, therethrough. In some embodiments, a partitioning unit may be held between a slit 140 in the wall of receptacle 110 and a passage 142 formed between a rod 144 and a block 146.

In some embodiments, receptacle 110 may include a plurality of partitioning units 150, configured to limit the flow of fluids from inlet 120 to access only selective locations at receptacle 110 and hence selective organ chips. Thus, partitioning units 150 may enable to manipulation of the flow within receptacle 110.

For example, and as shown in FIG. 3B, by using positioning several partitioning units 150 at the periphery of receptacle 110, the flow from inlet 120 may be confined to the periphery of receptacle 110, and thus cannot access the center of receptacle 150. This manipulation may provide a linear flow, and/or may mimic linear blood flow.

Alternatively, and as shown in FIG. 3C, by positioning several partitioning units 150 at the periphery of receptacle 110 and/or at the center of receptable 110, fluids from inlet 120 flow at the periphery, and also cross the center of receptacle 110. This manipulation may provide a non-linear flow, and/or may mimic non-linear blood flow.

In some embodiments, receptacle 110 may further comprise at least one reservoir 160 in fluid communication with the fluids within receptacle 110. In some embodiments, reservoir 160 may be enclosed within block 146, while in fluid communication with the fluids within receptacle 110.

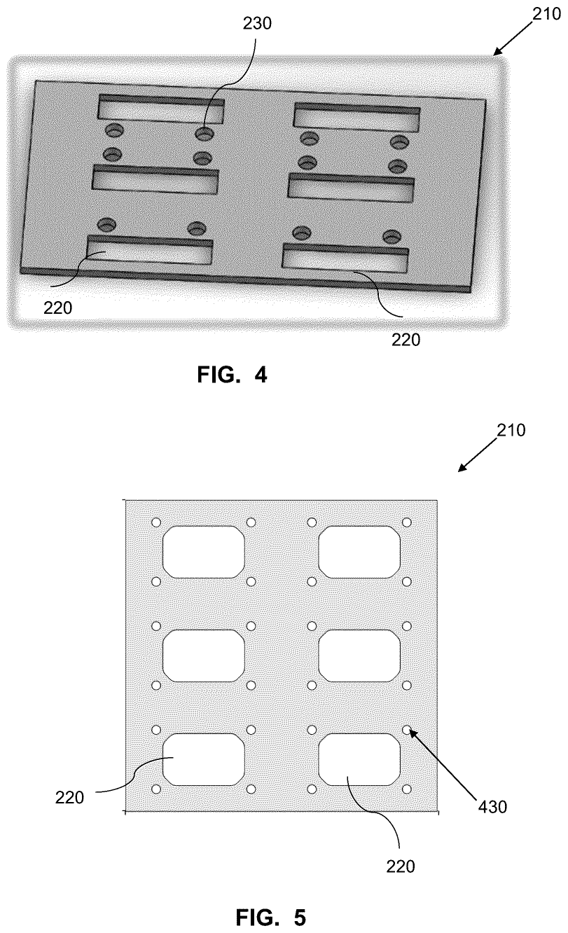

FIGS. 4 and 5 constitute a perspective view and a top view, respectively, of a cover layer 210, according to some embodiments. Optionally, the cover layer 210 may be configured to cover receptacle 110 or 440, non-hermetically, thereby allow gas exchange between receptacle 110 and the environment. In some embodiments, cover layer 210 may comprise a plurality of slots 220, wherein each slot 220 may be configured to hold one organ chip 310 such that the bottom part of an organ chip may be immersed in the fluids within receptacle 110 or 440, when full of fluids, the top part of organ chip 310 may stick out above the top surface of cover 210. When receptacle 110 may be filled with fluids, then organ chip 310 may be held by cover 210 such that the bottom side of membrane 320 may be contacting the fluids within receptacle 110.

In some embodiments, a cover layer 210 may further comprise a plurality of reservoir opening 230 which may be configured to be positioned above the at least one reservoir 160 thereby allow withdrawal of fluids from the receptacle through the at least one reservoir opening 230, and hence enabling withdrawal of fluids from receptacle 110 through reservoir opening 230.

In some embodiments, reservoir opening 230 may be blocked by plugs (not shown) when not in use, to prevent contamination of cartridge 100 and to avoid evaporation of fluids from receptacle 110.

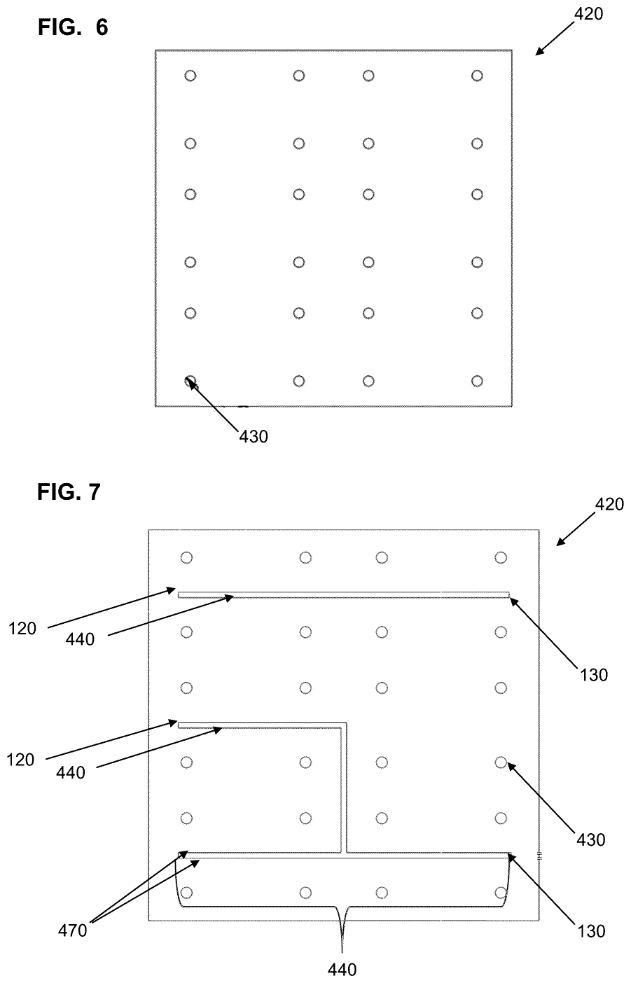

FIG. 6 constitutes a top view of a base layer 420, according to some embodiments. In some embodiments, the base layer 420 may include one or more holes 430 through which screws 460 may pass to connect the base layer 420 to the cover layer 210 and/or one or more cover sheets 410.

FIG. 7 constitutes a top view of a base layer 420 and one or more channels (receptacles) 440 through which the fluid passes. In an embodiment, the channels 440 may be affixed to the base layer 420. Optionally, the channels may comprise two members 470. Optionally, the members may be flexible, semi-flexible or rigid. Optionally, the members may be permeable, semi-permeable or impermeable. Optionally, the members 470 may be parallel to each other. Optionally, the distance between the members 470 may be predetermined. In an embodiment, the distance between the members 470 may be between 0.5 mm to 1 mm, and/or between 1 mm to 1.5 mm, and/or between 1.5 to 2 mm, and/or between 2 mm to 3 mm, and/or between 3 mm to 5 mm, and/or between 5 mm to 10 mm.

In an embodiment, the channels 440 may be open on both ends. Optionally, the ends of the channels 440 may be connected to one or more fluid reservoirs, e.g., an inlet 120 and an outlet 130 which may be configured to deliver and/or withdraw fluids, respectively. Optionally, the channels 440 may be open at the top (i.e., the base layer 420 and the flexible members 470 comprise 3 walls of a channel 440). Optionally, the top of a channel 440 may be sealed by a cover layer 210 and/or one or more organ chips 310 and/or one or more cover sheets 410.

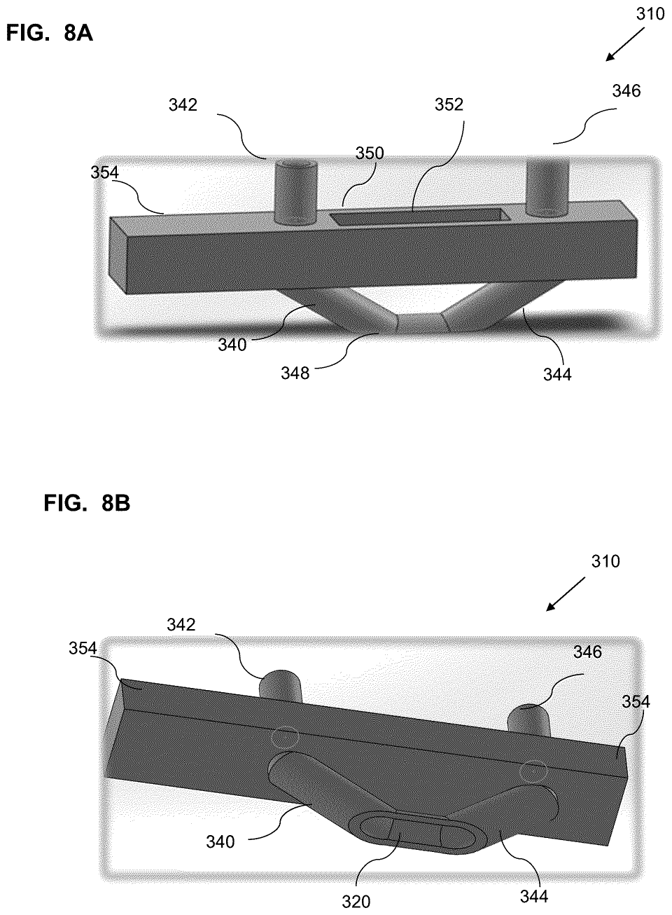

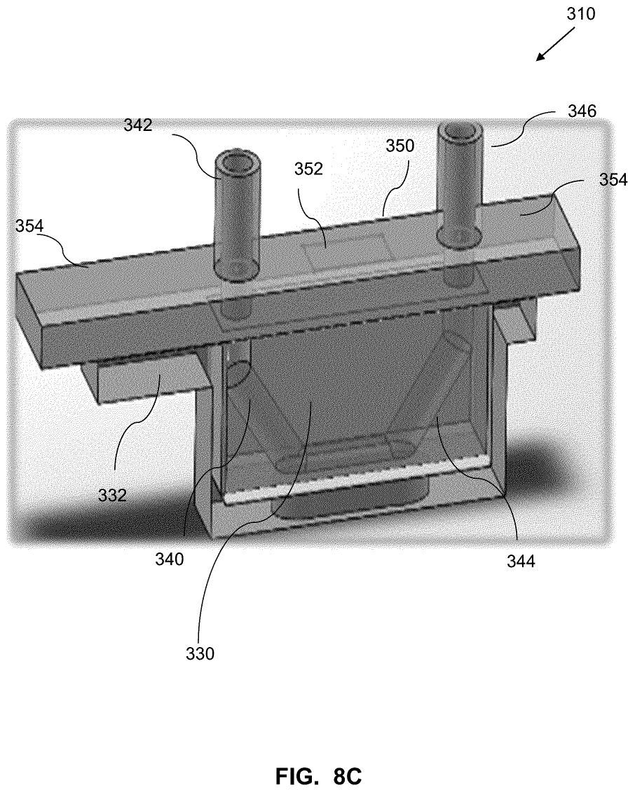

FIGS. 8A-8C constitute perspective views of organ chip 310, according to some embodiments. In some embodiments, organ chip 310 may include a bottom face 348 and a top face 350. In some embodiments, organ chip 310 may include a membrane 320 positioned at the bottom face 348. Optionally, the membrane may have an upper side and a lower side each configured for cell or organelle culture. Optionally, the membrane may be permeable to fluids.

In some embodiments, an organ chip 310 may further include an inlet conduit 340 having an external inlet 342 protruding through and/or open to the top face 350 of organ chip 310. Optionally, the inlet conduit 340 may be configured to deliver a second fluid into organ chip 310, and particularly to the top side of membrane 320.

In some embodiments, an organ chip 310 may further include an outlet conduit 344 having an external outlet 346 protruding through and/or open to the top face 350 of organ chip 310. Optionally, the outlet conduit 344 may be configured to allow withdrawal of fluids from organ chip 310.

In some embodiments, inlet conduit 340 and outlet conduit 344 may form a u-shaped structure. Optionally, conduits 340 and 344 may be the u-shape arms through which the second fluid flows, passing through bottom face 348 and particularly membrane 320.

In some embodiments, membrane 320 may be attached to the bottom of organ chip 310 by a fastener 332 (shown in FIG. 8C, detached from organ chip 310). In some embodiments, conduits 340 and 344 may be enveloped by cover 330 which may be configured to support the structure of organ chip 310.

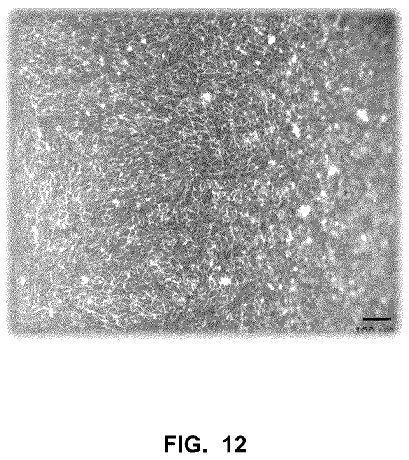

In some embodiments, top face 350 of organ chip 310 may further include a channel (not shown) extending from the upper side of membrane 320 to a window 352 at top face 350 of organ chip 310. Optionally, window 350 may be configured to enable view of the cells or organelles cultured on the upper side of membrane 320. FIG. 12 shows an image of cell culture grown on the upper side of membrane 320.

In some embodiments, the organ chip 310 may further include side arms 354 configured to stabilize organ chip 310 when settled in a cover layer 210, as discussed above.

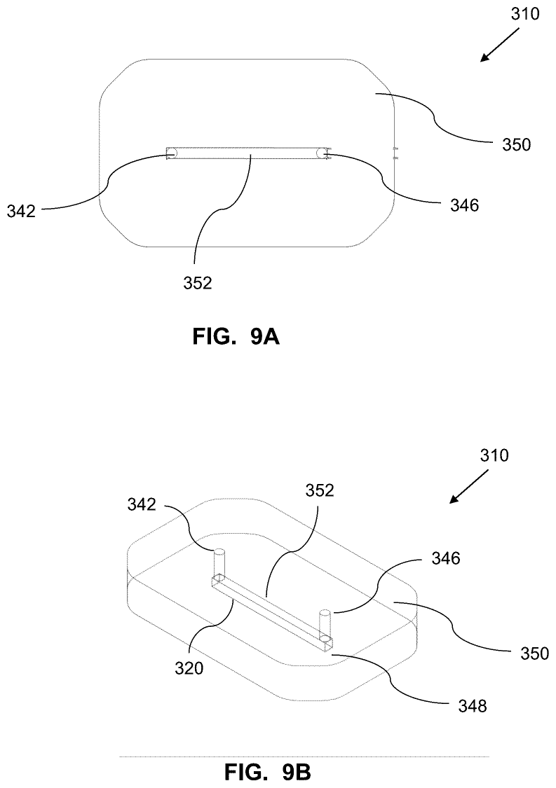

FIGS. 9A-9B constitute a top view and a perspective view of organ chip 310, respectively, according to some embodiments. In some embodiments, organ chip 310 may include a bottom face 348 and a top face 350. In some embodiments, organ chip 310 may include a membrane 320 positioned at the bottom face 348. Optionally, the membrane may have an upper side and a lower side. Optionally, each side may be configured for cell or organelle culture, Optionally, the membrane may be permeable to fluids.

In some embodiments, an organ chip 310 may further include an inlet conduit 340 having an external inlet 342 protruding through and/or open to the top face 350 of organ chip 310, wherein inlet conduit 340 may be configured to deliver a second fluid into organ chip 310, and particularly to the top side of membrane 320.

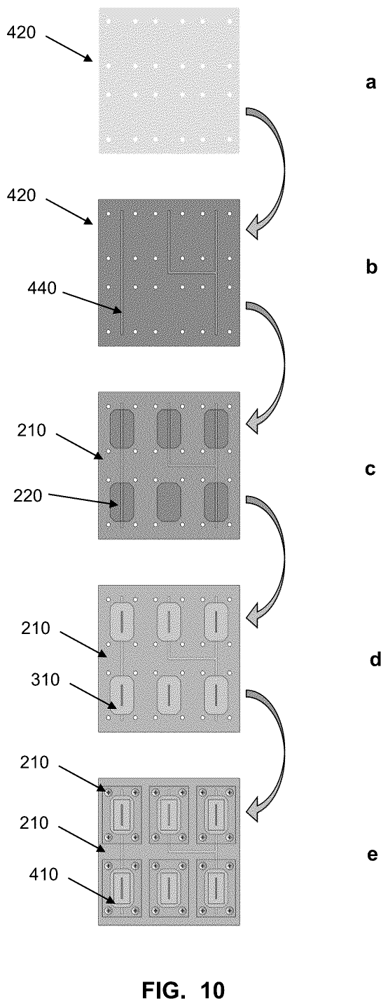

Reference is now made to FIG. 10, which constitutes a flow diagram describing an organ-on-a-chip system, in accordance with some embodiments. In an embodiment, a. shows a base layer 420 (e.g., made of poly(methyl methacrylate) (PMMA), polycarbonate (PC), and the like); b. to which adjustable flexible members 470 (e.g., made of polydimethylsiloxane (PDMS), natural rubber, thermoplastic elastomers, inorganic compounds, and the like) are attached (e.g., by gluing, molding, baking, binding, attaching, and the like) to produce one or more channels 440; c. A cover layer 210 comprising one or more openings 220 may be attached (for example, by screws through corresponding holes 430, or other suitable attachment means, such as grooves and hooks), to the base layer 420; d. one or more organ chips 310 may be inserted into openings 220 in the cover layer 210; and e. each organ chip 310 may be covered by a cover sheet 410 which may be attached to the cover layer 210 and/or base layer 420, and which may firmly presses the organ chip 310 down, such that the membrane 320 may be immersed in the fluid within the channels 440.

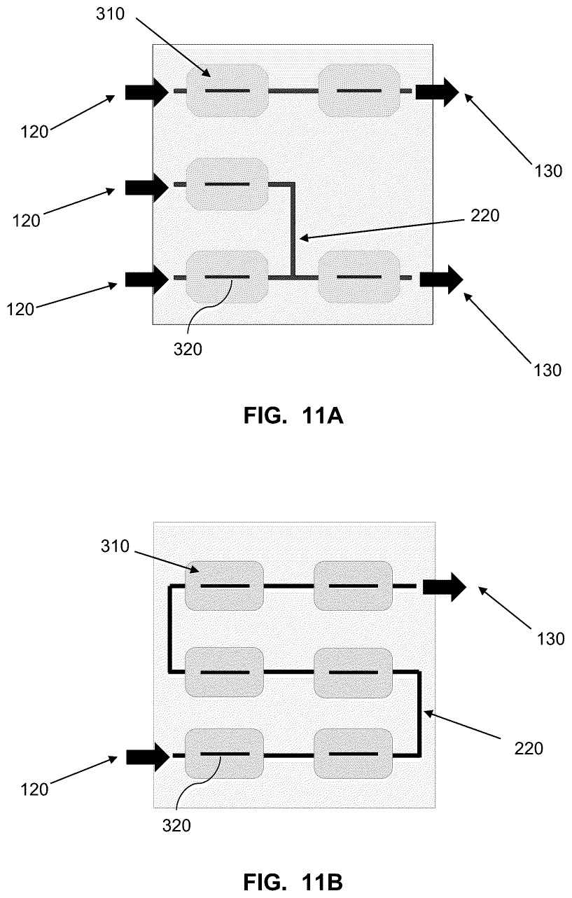

Reference is now made to FIGS. 11A-11B, which represent uses of an organs-on-a-chip system, in accordance with some embodiments. In an embodiment, FIG. 11A shows two organ chips in a row (e.g., in series) at the top, and shows two organ chips at the bottom in parallel followed by one organ. In an embodiment, FIG. 11B shows 6 organ chips connected in series.

Reference is now made to FIG. 12 which shows an image of cell culture grown on the upper side of a membrane of an organ chip, within a perfused cartridge, according to some embodiments.

In some embodiments, there is provided an organ-on-a-chip system, comprising at least one cartridge (device) as disclosed herein, and a plurality of pumps connected to the inlets and outlets of the receptacle and the organ chips, at least one of the pumps may be directed to deliver fresh fluids, in particular, fresh first fluid (e.g. blood or blood equivalent media or tissue culture medium) into receptacle, and at least one other pump may be directed to withdraw fluids from receptacle. Optionally, the system may also include one or more sources of fresh first fluids and/or fresh second fluids.

In some embodiments, there is provided a method for assembling a cartridge for culturing organs-on-a-chip, the method includes one or more of the steps of:
(a) providing a receptacle configured to contain fluids, the receptacle comprises at least one receptacle inlet configured to deliver a first fluid into the receptacle, at least one receptacle outlet configured to withdraw fluids from the receptable, and optionally a plurality of partitioning holders configured to hold partitioning units;
(b) providing a first container containing a first fluid and a second container containing a second fluid;
(c) fluidly connecting the first container to the at least one receptacle inlet, and fluidly connecting the at least one receptacle inlet to a first drainage container;
(d) providing at least one organ chip having a bottom face configured to be in fluid communication with fluids within the receptacle, the organ chip comprising
   i. a membrane located at the bottom face of the at least one organ chip, the membrane may have an upper side and a lower side each configured for cell or organelle culture, wherein the membrane may be permeable to fluids and wherein the upper side of the membrane may be facing the top face of the organ chip;
   ii. an inlet conduit having an external inlet protruding through the top face of the organ chip, wherein the inlet conduit may be configured to deliver a second fluid into the organ chip;
   iii. an outlet conduit having an external outlet protruding through the top face of the organ chip, wherein the outlet conduit may be configured to withdraw fluids from the organ chip,
(e) covering the receptacle with a cover layer comprising at least one slot configured to hold the at least one organ chip;
(f) positioning the at least one organ chip in the at least one slot such that the bottom face of the organ chip and the lower side of the membrane may be immersed in the receptacle, and the external outlet and inlet may protrude outside the cartridge, through the cover layer; and
(g) fluidly connecting the second container to the external inlet, and fluidly connecting the external outlet to a second drainage container,
wherein said fluidly connecting comprises connecting through conduits and pumps to initiate flow-in through the inlets and flow-out (drainage) through the outlets.

In some embodiments, the method may further comprise adding to the first container at least one pharmaceutically active ingredient. Optionally, a pharmaceutical composition may comprise the at least one pharmaceutically active ingredient. Optionally, the pharmaceutically active ingredient may be a antibiotics, sympathomimetics, antihistamines, anticholinergics, anti-inflammatory, corticosteroids, antiseptics, antifungals, antitussives, mast cell stabilizers, leukotriene antagonists, androgens, antiandrogens, gonadotropin, growth hormones, antidiabetics, thyroid hormones, antithyroid drugs, vasopressin analogues, cholinergics, antispasmodics, haemostatic drugs, antifibrinolytics, hormone replacement therapy (HRT), bone regulators, beta-receptor agonists, dopamine agonists, antileprotics, antituberculous drugs, antimalarials, anthelmintics, amoebicides, antivirals, antiprotozoals, probiotics, prebiotics, antitoxins, antivenoms, vaccines, immunoglobulins, immunosuppressants, interferons, monoclonal antibodies, cytotoxic drugs, therapeutic antibodies, sex hormones, aromatase inhibitors, somatostatin inhibitors, recombinant interleukins, G-CSF, erythropoietin, psychedelics, hypnotics, anaesthetics, antipsychotics, eugeroics, antidepressants (including tricyclic antidepressants, monoamine oxidase inhibitors, lithium salts, and selective serotonin reuptake inhibitors (SSRIs)), antiemetics, anticonvulsants, antiepileptics, anxiolytics, barbiturates, movement disorder (e.g., Parkinson's disease) drugs, nootropics, stimulants (including amphetamines), benzodiazepines, cyclopyrrolones, dopamine antagonists, antihistamines, cholinergics, anticholinergics, emetics, cannabinoids, and 5-HT (serotonin) antagonists, contrast agents, anticoagulants, heparin, antiplatelet drugs, fibrinolytics, anti-hemophilic factors, haemostatic drugs, hypolipidaemic agents, ACE inhibitors, angiotensin receptor blockers, beta-blockers, alpha blockers, calcium channel blockers, thiazide diuretics, loop diuretics, aldosterone inhibitors, calcium channel blockers, diuretics, cardiac glycosides, antiarrhythmics, nitrate, antianginals, vasoconstrictors, vasodilators, laxatives, antispasmodics, antidiarrhoeals, bile acid sequestrants, opioids, antiflatulents, antidopaminergics, proton pump inhibitors (PPIs), H2-receptor antagonists, cytoprotectants, prostaglandin analogues, etc.

In some embodiments, the receptacle may further comprise at least one reservoir in fluid communication with the fluids within the receptacle. Optionally, the cover layer may further comprise at least one reservoir opening positioned above the at least one reservoir thereby enabling withdrawal of fluids from the receptacle through the at least one reservoir opening

In some embodiments, the method may further comprise withdrawing (obtaining) a sample of fluids from the at least one reservoir. Optionally, withdrawing a sample of fluids from the at least one reservoir may be performed periodically. Optionally, the method may further include monitoring the amount or activity of at least one component in the sample.

In some embodiments, the volume of the withdrawn sample may be in the range of 10 to 200 μl, or any subranges thereof.

In some embodiments, various types of cells may be used with the systems disclosed herein. In some embodiments, the cells may be cell lines, tissue culture cells, primary cells, stem cells, organoids, and the like, or any combinations thereof. In some embodiments, the cells may include such cells as, but not limited to: vascular endothelial cells, cancerous cells, epithelial cells, alveolar epithelial cells, immune cells, enterocytes, and goblet cells, cardiac cells, muscle cells, adiposes cells, skin cells, hepatic cells, spheroids, stem cell-derived organoids, and the like, or any combinations thereof. Each possibility is a separate embodiment.

In some embodiments, the at least one component may be at least one pharmaceutically active ingredient. Optionally, periodical monitoring the amount of the at least one pharmaceutically active ingredient in samples may be performed. Optionally, monitoring the amount of the at least one pharmaceutically active ingredient may be further utilized for pharmacokinetic evaluation.

### EXAMPLES

### Example 1: Manufacture of an Organ Chip

SolidWorks CAD software was used to design a mold for fabrication of the organ chip. The mold was printed with a commercial polylactic acid filament using a Raise 3D Pro2 Dual Extruder 3D Printer (Raise Technologies, Inc.). Then, the molds were filled with polydimethylsiloxane (PDMS) prepared by mixing Sylgard 184® (Dow Corning, Midland, MI) with the curing agent at a ratio of 1:10, followed by curing at 60° C. for almost 4 h. The resulting PDMS were cleaned in ethanol, dried at room temperature (RT), and then activated in oxygen plasma (Atto-BR-200-PCCE, Diener Electronic, Germany).

Polycarbonate (PC) membranes (0.4 μm pore size, it4ip S.A., Belgium), 25 μm thick, were cut to size with their protective backing on. The protective backings were then removed, and the PC membranes were rinsed with isopropanol, dried under a stream of compressed air, and activated in oxygen plasma for 1 min (Diener Electronic, Germany). Then, the membranes were immersed for 30 min in 5% aqueous solution of 3-aminopropyltriethoxysilane (APTES, Sigma-Aldrich) in order to introduce amino groups at the surface of the PC membrane. Then they were washed three times with water and dried under a stream of compressed air.

PDMS block and PC membranes were then aligned and brought into contact, gently pressed together to ensure conformational contact, and baked at 60° C. there they were left overnight.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

1.-22. (canceled)

23. A cartridge for culturing organ-on a chip, comprising:
a. a receptacle configured to contain fluids, the receptacle comprises at least one receptacle inlet configured to deliver a first fluid into the receptacle, and at least one receptacle outlet configured to withdraw fluids from the receptable;
b. at least one organ chip having a bottom face configured to be in fluid communication with the fluids within the receptacle, the organ chip comprising
i. a membrane located at the bottom face of the at least one organ chip, the membrane having an upper side and a lower side each configured for cell, tissue or organoid culture, wherein the membrane is permeable to fluids and wherein the upper side of the membrane is facing the top face of the organ chip;
ii. an inlet conduit having an external inlet protruding through the top face of the organ chip, wherein the inlet conduit is configured to deliver a second fluid into the organ chip;
iii. an outlet conduit having an external outlet protruding through the top face of the organ chip, wherein the outlet conduit is configured to withdraw fluids from the organ chip; and
c. a cover layer configured to cover the receptacle, the cover layer comprising at least one slot configured to hold the at least one organ chip such that the bottom face of the organ chip and the lower side of the membrane are immersed in the fluid in the receptacle and the external outlet and inlet are open to or protrude outside the cartridge, through the cover layer.

24. The cartridge of claim 23, wherein the first fluid is blood or blood equivalent medium, and the second fluid is a tissue culture medium, body fluid other than blood, or an artificial equivalent thereof.

25. The cartridge of claim 23, wherein the receptacle further comprises at least one reservoir in fluid communication with the fluids within the receptacle, and wherein the cover layer further comprises at least one reservoir opening configured to be positioned above the at least one reservoir thereby enabling withdrawal of fluids from the receptacle through the at least one reservoir opening.

26. The cartridge of claim 23, wherein the receptacle further comprises at least one fluid impermeable partitioning unit creating a barrier that prevents flow of the fluids out of the receptacle.

27. The cartridge of claim 26, wherein the partitioning unit comprises flexible, semi-flexible or rigid members; or wherein the partitioning unit comprises rigid members which are held in place by partitioning holders.

28. The cartridge of claim 23, comprising a plurality of organ chips, wherein the cover layer comprises a plurality of slots, each configured to hold one organ chip from the plurality of organ chips.

29. The cartridge of claim 25, comprising a plurality of reservoirs, wherein the cover layer comprises a plurality of reservoir openings, each configured to be positioned above one reservoir from the plurality of reservoirs.

30. The cartridge of claim 23, wherein the top face of the organ chip comprises an opening configured to enable view of the cells or organelles cultured on the top side of the membrane.

31. The cartridge of claim 23, wherein the receptacle comprises a plurality of receptacle inlets and a plurality of receptacle outlets.

32. An organ-on-a-chip system, comprising:
at least one cartridge according to claim 23;
a container containing the first fluid fluidly connected to the receptacle through the at least one receptacle inlet;
a container containing the second fluid fluidly connected to the organ chip through the external inlet;
a first withdrawal pump configured to withdraw fluids from the receptacle through the at least one receptacle outlets; and
a second withdrawal pump configured to withdraw fluids from the organ chip through the external outlet.

33. The organ-on-a-chip system of claim 31, wherein the at least one cartridge comprises a plurality of organ chips.

34. A method for assembling a cartridge for culturing organs-on-a-chip, the method comprising the steps of:
a. providing a receptacle configured to contain fluids, the receptacle comprises at least one receptacle inlet configured to deliver a first fluid into the receptacle, at least one receptacle outlet configured to withdraw fluids from the receptable;
b. providing a first container containing a first fluid and a second container containing a second fluid;
c. fluidly connecting the first container to the at least one receptacle inlet, and fluidly connecting the at least one receptacle inlet to a first drainage container;
d. providing at least one organ chip having a bottom face configured to be in fluid communication with fluids within the receptacle, the organ chip comprising
i. a membrane located at the bottom face of the at least one organ chip, the membrane having an upper side and a lower side each configured for cell or organelle culture, wherein the membrane is permeable to fluids and wherein the upper side of the membrane is facing the top face of the organ chip;
ii. an inlet conduit having an external inlet protruding through the top face of the organ chip, wherein the inlet conduit is configured to deliver a second fluid into the organ chip;
iii. an outlet conduit having an external outlet protruding through the top face of the organ chip, wherein the outlet conduit is configured to withdraw fluids from the organ chip,
e. covering the receptacle with a cover layer comprising at least one slot configured to hold the at least one organ chip;
f. positioning the at least one organ chip in the at least one slot such that the bottom part of the membrane is immersed in the fluids within the receptacle and the external outlet and inlet protrude outside the cartridge, through the cover layer; and
g. fluidly connecting the second container to the external inlet, and fluidly connecting the external outlet to a second drainage container,
wherein said fluidly connecting comprises connecting through conduits and pumps to initiate flow-in through the at least one receptacle inlet and the external inlet and flow-out through the at least one receptacle outlet and the external outlet.

35. The method of claim 34, wherein the receptacle further comprises at least one reservoir in fluid communication with the fluids within the receptacle, and wherein the cover layer further comprises at least one reservoir opening positioned above the at least one reservoir thereby enabling withdrawal of fluids from the receptacle through the at least one reservoir opening.

36. The method of claim 34, further comprising the step of adding to the first container a pharmaceutical composition, comprising at least one pharmaceutically active ingredient.

37. The method of claim 34, further comprising the step of withdrawing a sample of fluids from the at least one reservoir.

38. The method of claim 37, further comprising monitoring the amount or activity of at least one component in the sample.

39. The method of claim 38, wherein the at least one component is the at least one pharmaceutically active ingredient, wherein periodical monitoring the amount thereof is further utilized for pharmacokinetic evaluation.

40. An organ chip having a bottom face configured to be in fluid communication with fluids within a receptacle, the organ chip comprising:
i. a membrane located at the bottom face of the organ chip, the membrane having an upper side and a lower side each configured for cell, tissue or organoid culture, wherein the membrane is permeable to fluids and wherein the upper side of the membrane faces the top face of the organ chip;
ii. an inlet conduit having an external inlet protruding through the top face of the organ chip, wherein the inlet conduit is configured to deliver a second fluid into the organ chip;
iii. an outlet conduit having an external outlet protruding through the top face of the organ chip, wherein the outlet conduit is configured to withdraw fluids from the organ chip.

41. The organ chip of claim 40, wherein the second fluid is a tissue culture medium, body fluid other than blood, or an artificial equivalent thereof.

42. The organ chip of claim 40, wherein the organ chip is for use with a cartridge for culturing organ on a chip.
